(19) United States
(12) Patent Application Publication
(10) Pub. No.: US 2022/0287633 A1
(43) Pub. Date: Sep. 15, 2022

(54) OPPORTUNISTIC RETRIEVAL OF ANALYTE VALUE TO IMPROVE AUTOMATIC DRUG CONTROL ACCURACY

(71) Applicant: Insulet Corporation, Acton, MA (US)

(72) Inventors: Yibin ZHENG, Hartland, WI (US); Ashutosh ZADE, San Diego, CA (US); Joon Bok LEE, Acton, MA (US)

(21) Appl. No.: 17/685,937

(22) Filed: Mar. 3, 2022

Related U.S. Application Data
(60) Provisional application No. 63/158,643, filed on Mar. 9, 2021.

(51) Int. Cl. A61B 5/00; A61B 5/145; A61M 5/172

(52) U.S. Cl. CPC A61B 5/4839; A61B 5/0002; A61B 5/14532; A61M 5/1723; A61M 2205/3561; A61M 2230/201; A61M 2205/3592; H04W 76/10

(57) ABSTRACT

Disclosed are processes and techniques implementable by devices and a drug delivery system to maintain optimal drug delivery for a user according to a user's diabetes treatment plan. The disclosed techniques enable a drug delivery device that received an analyte measurement value from an analyte sensor during a prior cycle time to determine a present analyte measurement value has not been received during a present cycle time. A processor of the drug delivery device may, in response to the determination, initiate an action to obtain the present analyte measure value from another device or an estimate of the present analyte measurement value. Based on an outcome of the initiated action, the processor may calculate a dosage of the drug using the present analyte measurement value or information related to the present analyte measurement value. The drug delivery device may also be operable to output the calculated dosage of the drug.

### RELATED APPLICATIONS

This application claims the benefit of U.S. Provisional Patent Application No. 63/158,643, filed Mar. 9, 2021, the contents of which are incorporated herein by reference in their entirety.

### BACKGROUND

An automated insulin delivery system relies on receiving correct blood glucose readings from blood glucose monitors or user inputs. Some automated insulin delivery systems are able to estimate and keep predicted values of a user's blood glucose readings based upon various parameters within the system. As the components of the automated insulin delivery systems become more specialized and compact, wireless communication between the respective components of the automated insulin delivery system has become less optimal for various reasons. Optimal wireless communications are affected by various factors including, but not limited to, tissue that obstructs radio frequency communication, the quality of communication components, varying power conditions, compatibility differences between manufacturers of the respective components, or the like. As a result, in some instances, the automated insulin delivery system may miss the transmission of a most recent blood glucose reading. This may cause the automated insulin delivery system to use a prediction of the missed most recent blood glucose reading in a determination of a next amount of insulin to be delivered. While use of the predicted suggested insulin may be nondetrimental for a short duration, as the number of missing points increases, the predicted values may, over longer durations, deviate from ground truth, and other fallback mechanisms may need to be used, such as delivering a conservation basal amount instead of a basal amount tailored to the user's actual current blood glucose level. As such, the efficacy of the automated insulin delivery system may be reduced.

### BRIEF SUMMARY

In an aspect, a drug delivery device includes a processor. The drug delivery device also includes a memory storing instructions that, when executed by the processor, configure the drug delivery device to receive an analyte measurement value from an analyte sensor during a prior cycle time, and determine a present analyte measurement value has not been received during a present cycle time, where the present analyte measurement value is received after the analyte measurement value received during the prior cycle time. In response to the determination, initiate an action to obtain the present analyte measurement value, and based on an outcome of the initiated action, a dosage of the drug using information related to the present analyte measurement value may be calculated. The drug delivery device may be further operable to output, or deliver, the calculated dosage of the drug.

In another aspect, a method is provided that includes receiving, by a drug delivery device configured to deliver a drug, an analyte measurement value from an analyte sensor during a prior cycle time, determining a present analyte measurement value has not been received during a present cycle time, in response to the determination, initiating an action to obtain the present analyte measurement value, and based on an outcome of the initiated action, calculating a dosage of the drug using information related to the present analyte measurement value. The method also includes delivering the calculated dosage of the drug.

In yet another aspect, a method is provided that includes receiving, by a processor of a drug delivery device, a series of analyte measurement values at predetermined intervals from an analyte sensor, generating an estimate of a next analyte measurement value in continuation of the series based on each of the analyte measurement values in the series, determining that the analyte sensor has not delivered the next analyte measurement value, querying other devices to determine if the other devices received the next analyte measurement value from the analyte sensor. After a predetermined period of time, an action to be taken related to delivery of a drug is determined, and a command signal based on the determination of the action is generated.

In a further aspect, a drug delivery system is provided that includes a controller having a processor, a communication device, and a memory, where the memory stores programming code and an automatic analyte control application. The drug delivery system also includes a drug delivery device having a processor, a communication device, a reservoir, and a pump mechanism. The drug delivery system also includes an analyte sensor having a processor, a communication device, a sensing/measuring device and a memory, the analyte sensor is configured to periodically obtain, via the sensing/measuring device, an analyte measurement value of an analyte of a user of the drug delivery system, where the drug delivery device is operable to receive an analyte measurement value from an analyte sensor during a prior cycle time, determine a present analyte measurement value has not been received during a present cycle time, in response to the determination, initiate an action to obtain the present analyte measurement value, and based on an outcome of the initiated action, and calculate a dosage of the drug using information related to the present analyte measurement value. The drug delivery device is further operable to output the calculated dosage of the drug.

### BRIEF DESCRIPTION OF THE DRAWINGS

To easily identify the discussion of any particular element or act, the most significant digit or digits in a reference number refer to the figure number in which that element is first introduced.

FIG. 1 illustrates an aspect of the disclosed subject matter in accordance with an embodiment.

FIG. 2 illustrates another aspect of the disclosed subject matter in accordance with another embodiment.

FIG. 3 illustrates yet another aspect of the subject matter in accordance with yet another embodiment.

FIG. 4 illustrates a process in accordance with an embodiment that is implementable by any of the aspects illustrated in FIGS. 1-3 and 6.

FIG. 5 illustrates another process in accordance with another embodiment that is implementable by any of the aspects illustrated in FIGS. 1-3 and 6.

FIG. 6 illustrates a functional block diagram of a system example suitable for implementing the example processes and techniques described herein.

### DETAILED DESCRIPTION

The following discussion describes various drug delivery system implementations and techniques for opportunistically obtaining information related to a present analyte measurement value that may not have been received by an automated analyte control (AAC) algorithm at a predicted time.

As described below, a drug delivery system may include a number of components, such as a drug delivery device, a controller and an analyte sensor. The drug delivery device may be controlled and operated based on signals containing information received from the analyte sensor. Control may be based on an application executing on the drug delivery device, the controller, the analyte sensor, or be distributed between two or all of the drug delivery device, the controller, or the analyte sensor.

An example of an automatic analyte control application or logic circuit may be specific to a particular type of analyte. Examples of analytes include blood glucose, a hormone, a protein, a medication, ketone, alcohol level, and the like. A control application specific to controlling a patient's blood glucose may be an Automatic Glucose Control (AGC) application that may rely on a blood glucose measurement value and other information that may be provided by a continuous glucose monitor (CGM). “Automatic glucose control” may refer to an algorithm or computer application that is operable to use blood glucose measurements to determine amounts of a drug to be delivered to a user to maintain the user's measured blood glucose within a set range of blood glucose measurement values. “Blood glucose measurement value” may refer to a numerical value representative of an amount of glucose in a user's blood that has the units of milligrams per deciliter (mg/dL). The other information provided by the CGM, such as past blood glucose measurement values, an indication of current trend of blood glucose measurements, and the like, may be included as part of, or separately from, the blood glucose measurement value. The AGC may be operable to estimate and store the estimated values of the user's blood glucose based upon various parameters within the drug delivery system. Such future trend information may be useful in case of missing CGM values to estimate suggested insulin for a short duration. As the number of missing points increases, the predicted values may deviate from ground truth and other mechanisms, such as implementing conservation basal delivery, may have to be used. Such fallback mechanisms, in case of missing blood glucose points, may impact accuracy of the AGC algorithm and thereby reduce the efficacy of the overall system.

The mechanisms and techniques described in this disclosure reduce the number of missing points by opportunistically attempting contact with other devices that may have received a CGM value which was missed by the device running the AGC algorithm. By opportunistically trying to retrieve a missed CGM value from other devices, the accuracy of the AGC algorithm can improve because the AGC algorithm is relying on actual/current blood glucose measurement values provided by the CGM as opposed to estimated blood glucose measurement values.

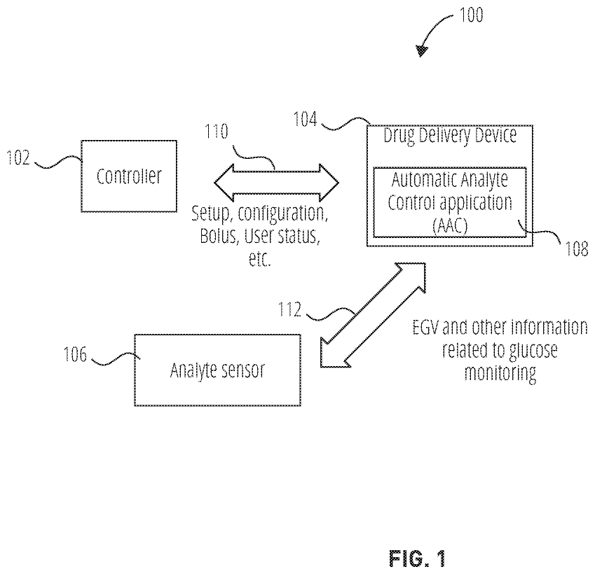

FIG. 1 illustrates an aspect of the subject matter in accordance with an embodiment of a drug delivery system. The drug delivery system 100 includes a controller 102, drug delivery device 104 and an analyte sensor 106. The controller 102 may communicate with the drug delivery device 104 via communication link 110 and the drug delivery device 104 may communicate with the analyte sensor 106 via communication link 112.

“Drug delivery system” may include a drug delivery device and an analyte sensor, such as a continuous glucose monitor, and, in some configurations, a controller and/or another device that receives data wirelessly from the analyte sensor. “Drug delivery device” may refer to a device configured to output a liquid drug to a user. The drug delivery device may respond to command signals received from a controller, which is a separate device. Alternatively, the drug delivery device may be configured with a processor that is not separate from the drug delivery device but is operable to perform functions as the controller. “Controller” may refer to a device having a processor that is configured to control operation of a drug delivery device and transmit and receive information related to measured blood glucose of a user associated with the drug delivery device and the controller. The functions performed by the controller may be executed by a processor integral to the drug delivery device. “Analyte sensor” may refer to a sensing and measuring device that may be configured to measure one or more of lactate, ketones, uric acid, sodium, a protein, potassium, alcohol levels, hormone levels, blood glucose, a medication, or the like.

In some examples, the analyte sensor 106 may be configured as a continuous glucose monitor, which may refer to a wearable device that may at least include a sensor configured to measure blood glucose of a wearer, a transmitter to transmit blood glucose measurement values measured by the sensor, and logic circuitry that controls the sensor and the transmitter. The analyte sensor 106 may be configured, as shown in another figure, with a processor, a communication device, a memory and a sensing/measuring device. The analyte sensor 106 may output analyte measurement values to any devices that are communicatively coupled to the analyte sensor 106 when operating properly (e.g., full power supply, established wireless connectivity, sensing/measuring device operation is proper, and whatever other functions enable the analyte sensor to detect and output an analyte measurement value). In the drug delivery system of FIG. 1, the analyte sensor 106 and the drug delivery device 104 may be communicatively coupled to one another, and the controller 102 and drug delivery device 104 may be communicatively coupled to one another. For example, the controller 102 may be wirelessly coupled to the drug delivery device 104 via communication link 110 and the drug delivery device 104 may be wirelessly coupled to the analyte sensor 106 via communication link 112. “Communication link” may refer to a signal pathway between a first device and a second device. As described below, information may be exchanged between the first device and the second device where both devices may transmit and receive information. In the example of FIG. 1, the analyte sensor 106 is not shown as communicating directly with the controller 102.

The analyte sensor 106 may provide an analyte measurement value at a specific time or time interval during an operational time period to the drug delivery device 104. An “analyte measurement value” may refer to a numerical value representative of an amount of an analyte being monitored by an analyte sensor and that has the units in common usage of the analyte being monitored, such as milligrams per deciliter (mg/dL), a percentage, or the like. In an example, the analyte sensor 106 may be configured to output an analyte measurement value at a predetermined cadence (e.g., every 5, 6, 8 or 10 minutes) or once within a set period of time (e.g., at time=0 in a repeating 5 minute period of time). The set period of time in the example of a diabetes treatment plan may be referred to as a “cycle” or “cycle time,” which may last 5 minutes, or about 5 minutes, about 3 minutes, or about 10 minutes, or the like. A specific time during the operation may be at the beginning of the operational time period.

The outputted analyte measurement value is intended, in this embodiment, for receipt by the automatic analyte control (AAC) application 108 executing on the drug delivery device 104, but may be shared with other applications, such as an automatic medication delivery application, such as an artificial pancreas application, or the like. The analyte measurement values may include information that includes an analyte measurement value, such a blood glucose measurement value, but also information such as analyte rate of change, status information related to a sensing/measuring device in the analyte sensor 106, past analyte measurement values (e.g., the past two or three analyte measurement values), and the like.

The controller 102 may be configured, as shown in other figures, with a processor, a memory, a user interface, a communication device, and the like. The memory may store information, such as previous analyte measurement values, delivered drug dosages, and the like. Additionally, the memory may store computer applications, such as a medication delivery application (MDA), an automatic analyte control application, and the like, that are executable by the processor and configure the processor to perform different functions as discussed herein. An “automatic analyte control application” may refer to a computer application that is operable to execute an algorithm operable to use analyte measurement values, including blood glucose measurement values, to determine amounts of a drug to be delivered to a user.

The controller 102 when executing the MDA may be configured to set operational parameters for the drug delivery device 104, such as user preferences for drug deliveries (e.g., times, basal and bolus) and notifications/alarms, drug delivery limits, user statistics (e.g., height, weight, gender, age, type of diabetes, or the like) as well as other settings.

The drug delivery device 104 of drug delivery system 100 may also be configured, as shown in other figures, with a processor, a memory, a user interface, a communication device, and the like. The memory may store information, such as previous analyte measurement values, delivered drug dosages, and the like. Additionally, the memory may store computer applications, such as a medication delivery application (MDA), automatic analyte control application 108, and the like, that are executable by the processor and configure the drug delivery device 104 to perform different functions as discussed herein. The drug delivery device 104 may during a cycle be configured to use the present analyte measurement value and information related to the present analyte measurement value to determine a dosage of a drug to be delivered during the cycle. The dosage of the drug may be a basal dosage, a bolus dosage, or a combination of both. “Present analyte measurement value” may refer to the analyte measurement value that is generated by the analyte sensor during a cycle of the drug delivery system. “Information related to the present analyte measurement value” may refer to additional data that is related to the present analyte measurement value such as trend information (i.e., whether the measurement value is increasing, decreasing or level over a period of time), prior analyte measurement values, analyte sensor status (e.g., power level, occlusion status, or the like) and other information.

In the drug delivery system 100, the analyte sensor 106 may be configured to provide a present analyte measurement value to the drug delivery device 104. The present analyte measurement value may include information related to the present analyte measurement value as well as information related to past analyte measurement values. For example, when the analyte sensor 106 is a continuous glucose monitor (CGM), the analyte sensor 106 may be configured to provide a blood glucose measurement value. In addition, the analyte sensor 106, when configured as a CGM, may provide a blood glucose trend indication, as well as blood glucose measurement values from the past one, two, or more cycles. For example, if the CGM is configured to provide a blood glucose measurement value once every 5 minutes (i.e., once every cycle) to the drug delivery device 104, the CGM may provide a present analyte measurement value (i.e., a present blood glucose measurement value) at the beginning of a 5 minute cycle as well as past analyte measurement values (i.e., past blood glucose measurement values) from 5 minutes ago and 10 minutes ago. In the example, the present blood glucose measurement value may be provided at time t(0) and the past blood glucose measurement values may be from time t(−5) and time t(−10). The past blood glucose measurement values are obtained at a time prior to the present blood glucose measurement value time.

The automatic analyte control application 108 of the drug delivery device 104 may be configured to receive the respective analyte measurement value from the analyte sensor 106 during each respective cycle time, including prior cycle times and the present cycle time. In an operational example, the processors in each of the controller 102, the drug delivery device 104 and the analyte sensor 106 may be synchronized to a common clock. The drug delivery device 104 may be provided with information related to the analyte sensor 106 by an application executing on the controller 102. For example, a user may input an identifier of the analyte sensor 106 into a user interface presented on the controller 102. The drug delivery device 104 may receive the analyte sensor 106 identifier via communication link 110 from the controller 102. The drug delivery device 104 may use the analyte sensor 106 identifier to establish communication link 112 with the analyte sensor 106. For example, in some handshaking protocols, the drug delivery device 104 may provide its identifier to the processor of the analyte sensor 106. In this example, all analyte measurement values from the analyte sensor 106 are delivered to the drug delivery device 104.

Using known handshaking protocols or similar procedures, the communication link 112 may be established by the drug delivery device 104 and/or the analyte sensor 106 at the beginning of each cycle (e.g., preset time period) for a duration long enough to deliver a present analyte measurement value as well as information related to the present analyte measurement value. Once the present analyte measurement value and the information related to the present analyte measurement value are delivered, the communication link 112 may be dropped (i.e., disconnected) until the beginning of the next cycle. A purpose for reestablishing the communication link 112 at the beginning of every cycle may be to conserve energy for both the drug delivery device 104 and the analyte sensor 106.

A risk in having to reestablish the communication link 112 at every cycle is the potential to fail to establish the link, a delay in establishing the link that extends beyond the time of transmission of analyte measurement value(s) from the analyte sensor 106, or the like. Even if the communication link 112 is not reestablished at every cycle, there is still the potential for interference with the transmission due to movement of the user, presence of an electromagnetic or radio frequency noise source, or the like. For example, both the drug delivery device 104 and the analyte sensor 106 may be wearable devices that communicate most efficiently when in line of sight of one another. Placement of a drug delivery device 104 on an upper-left arm of a user and placement of the analyte sensor 106 on a right-side of the user's abdomen may not always provide the optimum line of sight placement. Of course, other placements of the respective devices may also result in limited line-of-sight between the devices.

In an example, the memory of the drug delivery device 104 may be configured to store an MDA, such as an artificial pancreas application or the automatic analyte control application 108, that is operable to use previous analyte measurement values and the information related to the previous analyte measurement values in an estimate of a value of a next analyte measurement value and store it in memory. In the event the drug delivery device 104 determines a present analyte measurement value has not been received during a present cycle time, the drug delivery device 104 may initiate an action to retrieve the estimated next analyte measurement value (based on prior received analyte measurement values) from the memory for use as the present analyte measurement value, which was missed.

In another example, the drug delivery device 104 may be configured to forward the present analyte measurement value and information related to the present analyte measurement value to the controller 102. The controller 102 may execute an MDA, such as an artificial pancreas application, that is operable to use the forwarded present analyte measurement value and information related to the present analyte measurement value in an estimate of a value of a next analyte measurement value.

In an example, the controller 102 may be configured to make an estimate of the present analyte measurement value. The drug delivery device 104 may initiate an action to obtain the present analyte measurement value in the event the drug delivery device 104 determines a present analyte measurement value has not been received during a present cycle time. Additionally, or alternatively, as the initiated action, the drug delivery device 104 may establish communication link 110 with the controller 102 and request the estimate of the next analyte sensor value. Using the estimate of the next analyte sensor value, the drug delivery device 104 may, for example, calculate a dosage of the drug using information related to the present analyte measurement value, and deliver the calculated dosage of the drug.

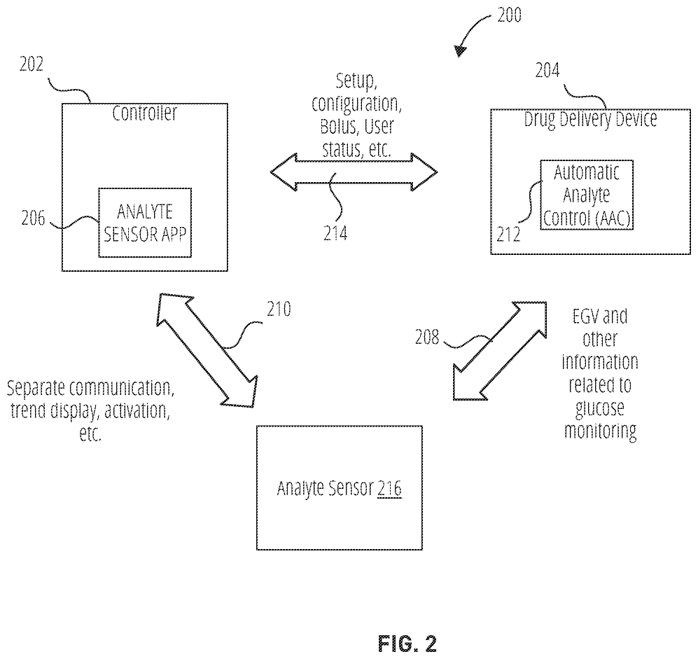

FIG. 2 illustrates another aspect of the subject matter in accordance with another embodiment. The drug delivery system 200 of FIG. 2 includes a controller 202, drug delivery device 204 and analyte sensor 216.

The controller 202 may be configured, as shown in other figures, with a processor, a memory, a user interface, a communication device, and the like. The memory may store information, such as previous analyte measurement values, delivered drug dosages, and the like. Additionally, the memory may store computer applications, such as a medication delivery application (MDA), automatic analyte control application, and the like, that are executable by the processor and cause the processor to perform different functions as discussed herein.

The drug delivery device 204 of drug delivery system 200 may also be configured, as shown in other figures, with a processor, a memory, a user interface, a communication device, and the like. The memory may store information, such as previous analyte measurement values, delivered drug dosages, and the like. Additionally, the memory may store computer applications, such as a medication delivery application (MDA), automatic analyte control application 212, and the like, that are executable by the processor and cause the drug delivery device 204 to perform different functions as discussed herein. The drug delivery device 204 may during a cycle be configured to use the present analyte measurement value and information related to the present analyte measurement value to determine a dosage of a drug to be delivered during the cycle. The dosage of the drug may be a basal dosage, a bolus dosage, or a combination of both.

The controller 202 may include an analyte sensor application 206 and the drug delivery device 204 may include an automatic analyte control application 212. The analyte sensor 216 operates and performs functions in the same manner as described with reference to analyte sensor 106 of FIG. 1.

The controller 202, drug delivery device 204 and analyte sensor 216 may each include communication devices and may be configured to exchange two-way communication signals between one another (e.g., the controller 202 can both receive signals from and send signals to either drug delivery device 204 or analyte sensor 216). Similarly, drug delivery device 204 may receive signals from and send signals to controller 202 and analyte sensor 216, and analyte sensor 216 may receive signals from and send signals to controller 202 and drug delivery device 204). The two-way communications may be over communication link 208, communication link 210 or communication link 214 depending upon which devices are communicating.

In an operational example, the analyte sensor application 206 in the controller 202 may facilitate activation of the analyte sensor 216, which may include providing identifiers of the controller 202 and the drug delivery device 204 to the analyte sensor 216. In addition, the analyte sensor application 206 may enable the controller 202 to set different parameters for the analyte sensor 216, such as alarm/notification messages and timing, communication protocols and the like, and display (numerically and/or graphically) information related to blood glucose measurement values, such as trend information, past and present blood glucose measurement values, and the like.

The analyte sensor application 206 may also direct the analyte sensor 216 to send analyte measurement values not only to the drug delivery device 204, but also to the controller 202 for retention in a memory (shown in another figure) of the controller 202. “Analyte sensor application” may refer to a computer application executed by circuitry of an analyte sensor that may be configured to control measurement and output at least an analyte measurement value at a predetermined cadence. In addition, the analyte sensor application may be configured to determine analyte measurement value trend information, maintain past analyte measurement values and the generation of messages or signals for output to at least one of a drug delivery device, a controller, a display device, a smart accessory device, or other devices, such as an additional continuous glucose monitor tracking device. “Additional continuous glucose monitor tracking device” may refer to a wearable device that includes sensors that are configured to measure blood glucose of a wearer of the tracking device. For example, the analyte sensor application 206 may configure the analyte sensor 216 to establish communication connections with both the controller 202 and the drug delivery device 204, such as communication link 210 and communication link 208. The communication device of the analyte sensor 216 may be configured to establish the communication links 208 and 210 at, or near (i.e., within milliseconds of), the beginning of each cycle. The analyte sensor application 206 may also be configured to communicate with the automatic analyte control application 212 and may provide a notification to the automatic analyte control application 212 that the analyte sensor application 206 is operable to receive analyte measurement values from the analyte sensor 216.

In a further example, the automatic analyte control application 212 executed by a processor of the drug delivery device 204 may determine that a present analyte measurement value including information related to the present analyte measurement value has not been received within the predetermined period of time. In response to the determination, the automatic analyte control application 212 may be configured to initiate actions to obtain the present analyte measure value, if possible.

The automatic analyte control application 212 may, in response to the determination that the present analyte measurement value has not been received, be configured to confirm a communication connection with the analyte sensor 216. The communication connection may be communication link 208. The automatic analyte control application 212 may use known methods to confirm that communication link 208 remains an established communication connection. Upon confirmation of the communication connection with the analyte sensor 216, the automatic analyte control application 212 may request that the analyte sensor 216 send the present analyte measurement value. In response, the automatic analyte control application 212 may receive an indication that the present analyte measurement value is not available in response to the request. For example, the present analyte measurement value may not be available because the present analyte measurement value is not retained in a memory of the analyte sensor 216, the period of time during which the present analyte measurement value is retained has passed, or the like.

In response to the present analyte measurement value being unavailable, the automatic analyte control application 212 executing on the drug delivery device 204 may establish a communication connection with the controller 202 and the analyte sensor application 206 that is communicatively coupled to the analyte sensor 216. Upon establishing the communication connection with the controller 202, the automatic analyte control application 212 may request the present analyte measurement value from the controller 202 and may receive the present analyte measurement value in response to the request.

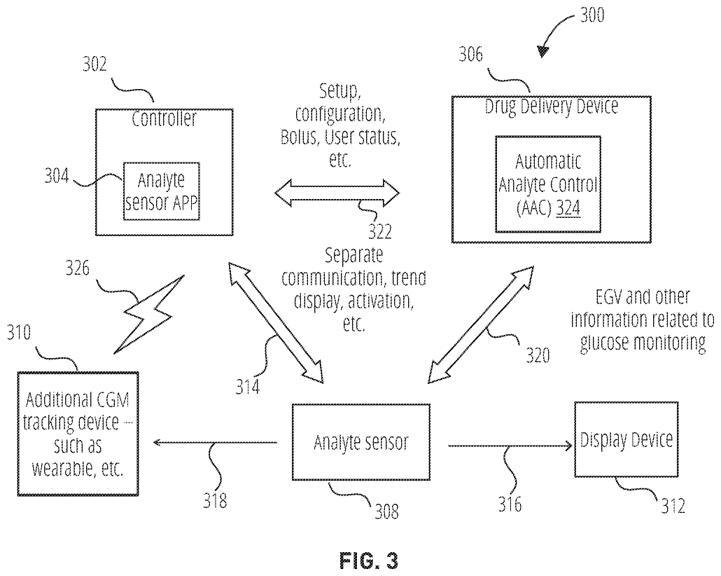

FIG. 3 illustrates yet another aspect of the subject matter in accordance with yet another embodiment. In an extension of drug delivery system 200 of FIG. 2, the drug delivery system 300 may include a controller 302, a drug delivery device 306 and an analyte sensor 308 as well as additional components, such as an additional continuous glucose monitor tracking device 310 and a display device 312 in communication with the analyte sensor 308. “Continuous glucose monitor display device” may refer to a display device having circuitry configured to present glucose measurement values transmitted from a continuous glucose monitor. “Additional continuous glucose monitor tracking device” may refer to a wearable device that includes sensors that are configured to measure blood glucose of a wearer of the tracking device.

The controller 302 may be configured, as shown in other figures, with a processor, a memory, a user interface, a communication device, and the like. The memory may store information, such as previous analyte measurement values, delivered drug dosages, and the like. Additionally, the memory may store computer applications, such as a medication delivery application (MDA), automatic analyte control application, and the like, that are executable by the processor and configure the processor to perform different functions as discussed herein.

The drug delivery device 306 of drug delivery system 300 may also be configured, as shown in another figure, with a processor, a memory, a user interface, a communication device, and the like. The memory may store information, such as previous analyte measurement values, delivered drug dosages, and the like. Additionally, the memory may store computer applications, such as a medication delivery application (MDA), automatic analyte control application 324, and the like, that are executable by the processor and configure the drug delivery device 306 to perform different functions as discussed herein. During a cycle, the drug delivery device 306 may be configured to use the present analyte measurement value (e.g., present blood glucose measurement value) and information related to the present analyte measurement value (e.g., information related to the present blood glucose measurement value) to determine a dosage of a drug to be delivered during the cycle. The dosage of the drug may be a basal dosage, a bolus dosage, or a combination of both.

Additional devices 310 may include an application device, a consumer type of device that may be an exercise tracker, a health tracker, a medical device, a tablet computer, a dedicated display device, a wearable device such as a smartwatch, a pendant smart device, a smartphone, smart speakers, home digital assistant, a laptop computer, a desktop computer, and the like. In some examples, the additional devices 310 may be an additional continuous glucose monitor tracking device and may be configured to communicate with the analyte sensor 308 when it is implemented as a continuous glucose monitor. The analyte sensor 308 may transmit blood glucose measurement values to the additional continuous glucose monitor tracking device 310. The additional continuous glucose monitor tracking device 310 may be configured to store the received blood glucose measurement values and information related to the blood glucose measurement values (e.g., trend indication, previous measurement values), display the blood glucose measurement values, and perform calculations, such as bolus calculations or the like based on the blood glucose measurement values and related information.

The controller 302, drug delivery device 306 and analyte sensor 308 of drug delivery system 300 may communicate via respective two-way communication links 314, 320 and 322. The additional continuous glucose monitor tracking device 310 and display device 312 may be configured to receive analyte measurement values from the analyte sensor 308 via information link 316 or information link 318. “Information link” may refer to a one-way signal pathway from one device to another. Information is sent from a transmitting device to a receiving device. If a two-way signal path, the receiving device may or may not provide an acknowledgement of receipt of the information without providing any further information. The additional continuous glucose monitor tracking device 310 may be configured to wirelessly communicate with the controller 302 and drug delivery device 306 via wireless communication link 326. “Wireless communication link” may refer to a signal transmission pathway that may be established using radio frequency, optical, or skin conductance signal transmissions between one or more devices in a drug delivery system environment. The additional continuous glucose monitor tracking device 310 may be a device specific to the analyte sensor 308 being implemented as a continuous glucose monitor (CGM).

In an operational example, the analyte sensor 308 may be operable to output an analyte measurement value and information related to the analyte measurement value. When implemented as a CGM, the analyte sensor 308 may output a blood glucose measurement value (which may be the present analyte measurement value in the examples) and information related to the blood glucose measurement value, which may include trend information, time stamp, previous blood glucose measurement values, or the like. The analyte sensor 308 may output the blood glucose measurement value via communication links 314 and 320 and information link 318 for receipt by the controller 302, the drug delivery device 306, the additional continuous glucose monitor tracking device 310, respectively. The blood glucose measurement value may be from the present cycle of the analyte sensor 308.

When the blood glucose measurement value transmitted by the analyte sensor 308 is received at the drug delivery device 306, the Automatic Analyte Control (AAC) 324 may process the blood glucose measurement value to determine a dosage of a drug, such as insulin, glucagon, a glucagon-like peptide, pramlintide, or a combination of two or more thereof, to be delivered to a user during the present cycle or a next cycle. In addition, the Automatic Analyte Control (AAC) 324 may use the received blood glucose measurement value to estimate a future or next blood glucose measurement value that is expected to be transmitted by the analyte sensor 308 at the next cycle (in the future).

However, there may be instances when the analyte sensor 308 is unable to deliver the present analyte measurement value for the present cycle to the drug delivery device 306. For example, the communication device of the analyte sensor 308, the communication device of the drug delivery device 306 or both may be unable to complete the handshaking protocol used to establish communication link 320. The processor (shown in another example) of the drug delivery device 306 may have procedures to follow when it determines that a present analyte measurement value has not been received.

For example, the Automatic Analyte Control (AAC) application 324 may have a prioritized table of which devices to query. In an example, the Automatic Analyte Control (AAC) 324 may first contact a CGM, which may be the analyte sensor 308, and if there is no response or the present analyte measurement value is unavailable, the controller 302 may be contacted second. In this example, the analyte sensor application 304 may be a continuous glucose monitor application that may be configured to receive the request from the Automatic Analyte Control (AAC) 324 for the present analyte measurement value and information related to the present analyte measurement value. “Continuous glucose monitor application” may refer to an application executed by circuitry of an analyte sensor that may be specifically configured to control measurement and output at least a blood glucose measurement value at a predetermined cadence. In addition, the continuous glucose monitor application may be configured to determine blood glucose measurement value trend information, maintain past blood glucose measurement values and the generation of messages or signals for output to at least one of a drug delivery device, a controller, a display device, an additional continuous glucose monitor tracking device, or a smart accessory device. Should the controller 302 not be able to satisfy the request, the Automatic Analyte Control (AAC) 324 may attempt to contact the additional continuous glucose monitor tracking device 310 via wireless communication link 326 to request the present analyte measurement value and information related to the present analyte measurement value.

If there is no response from the other devices (e.g., the controller 302 or additional continuous glucose monitor tracking device 310) or if no present analyte measurement value has been received in response to the request to the analyte sensor 308, the Automatic Analyte Control (AAC) application 324 may make an estimate based on previous analyte measurement values or use an existing estimate of the present analyte measurement value and an information related to the present analyte measurement value (e.g., trend information, rate of change, or the like) that was made based on previously received analyte measurement values and information related to the previous analyte measurement values. If the Automatic Analyte Control (AAC) 324 is using an existing estimate, the Automatic Analyte Control (AAC) application 324 may use an estimate that was stored in a memory of the drug delivery device 306 or received in response to a request sent to the controller 302.

In a further example, the drug delivery device 306 will try to talk to CGM, then controller, then wearable watch. All of the devices are synchronized to maintain a continuity of data. In addition, at setup identifiers of the drug delivery device, the CGM, the controller and the CGM tracking devices are shared within the drug delivery device system 300. In addition, security protocols may be in place to ensure that the communications remain confined to the devices in the drug delivery system 300.

In an alternative example, any device that receives the CGM data may transmit the received CGM data to the drug delivery device 306 to ensure that the present analyte measurement value is received by the drug delivery device 306. In such an instance, the processor of the drug delivery device 306 may analyze a time stamp of each message received from a respective device to determine whether the received message includes the present analyte measurement value. The analysis may confirm that the time stamp corresponds to an expected time that the present analyte measurement value was to be transmitted or obtained.

In an example, the automatic analyte control application 324, which may be an automatic glucose control application, may be configured to implement a stratified missing point compensation process. A missing point compensation process may be a process in which if a data point, such as the present analyte measurement value or information related to the present analyte measurement value is determined to be missing, the Automatic Analyte Control (AAC) 324 may take different actions. In an example, if a most recent blood glucose measurement value was below a preset minimum blood glucose measurement value level, such as 60 mg/dL, 70 mg/dL, 80, mg/dL or the like, the action taken by the drug delivery device 306 may be to suspend delivery of insulin (both basal and bolus doses). The drug delivery device 306 may continue to suspend delivery of insulin until a blood glucose measurement value is received. When, after the last suspension decision by the drug delivery device 306, a message is received from the analyte sensor 308 indicating that the blood glucose measurement value is now above the preset minimum blood glucose measurement value level, such as 60 mg/dL, the suspend command is removed because based on the most recently received blood glucose measurement value, the user's blood glucose is above 60 mg/dL.

In another embodiment, when a blood glucose measurement value is determined to be missing, predicted or estimated points may be substituted. For example, BG may be 300, then 310, then 320, but then have missing BG for a next point, but based on the trend the system can predict that BG is going to be 330 for the missing BG value. However, soon the prediction algorithm may lose confidence in the prediction. The processor may decide to be conservative and determine a basal dosage is appropriate and may output a basal dosage that may be slightly above (e.g., 3%, 5%, 7% or the like) a typical basal dosage.

Alternatively, in an example, the processor may not receive a blood glucose measurement value for 6 cycles, but in the 7th cycle, it may be determined, based on blood glucose measurement values received from other devices, that 3 blood glucose measurement values in the missing 6 cycles were greater than 300 mg/dL. In the example, the blood glucose measurement values for the missed 6 cycles may be received from another source such as a cloud-based service, an analyte sensor application on a controller or smartphone, or the like, instead of the CGM. Additionally, the automatic glucose control algorithm may use the received information to confirm the predictions. Based on confirmation that the predictions were within an allowable threshold of the measurement data values, the processor may determine that the delivery of the basal dosages may continue based on the predictions.

In a further example, the controller 302, the drug delivery device 306 or both may be operable to predict blood glucose measurement values several cycles ahead, such as 3-6 cycles into the future. These predictions of later blood glucose measurement values may be updated when the missing data is obtained. For example, the processor may be limited to relying on estimated blood glucose values for a set number of cycles, such as 3, 4, 5, or 6 cycles. Based on the last blood glucose measurement value that was not missed and the estimated blood glucose measurement values, the controller 302 or drug delivery device 306 may take further action. For example, the initial predicted values may include values of 300 mg/dL, 310 mg/dL and 320 mg/dL, but the updated predictions may be 330 mg/dL, 340 mg/dL and 350 mg/dL; however, after a 3rd cycle in which a present blood glucose measurement value has not been received from either the analyte sensor 308 or another device (e.g., controller 302 or additional continuous glucose monitor tracking device 310), trust in the predictions may decrease and the drug delivery device 306 may be configured to take different actions. In most examples, the analyte sensor 308, when configured as a continuous glucose monitor provides at least the past two measurement values (e.g., t=−10 minutes and t=−5 minutes) and the present blood glucose measurement value (e.g., t=0 minutes) to any external device to which it is sending information or in response to a request.

In a further example, rather than directly modifying the estimated system states, newly available valid readings from previous cycles can be incorporated as an envelope of high confidence of current predictions—specifically, the predictions that are utilized in the present cycle can be bounded within a certain numerical distance of the most recent newly available valid analyte measurement value.

Additionally, or alternatively, the drug delivery device 306 may have a processor operable to implement logic that modifies the actions taken based on the number of predictions used. As more predictions are being used to calculate drug dosages, the processor of the drug delivery device 306 may become more conservative in the delivery of the drug. For example, if the blood glucose measurement value is above 60 mg/dL, the processor of the drug delivery device 306 may take the action of not delivering any further drug.

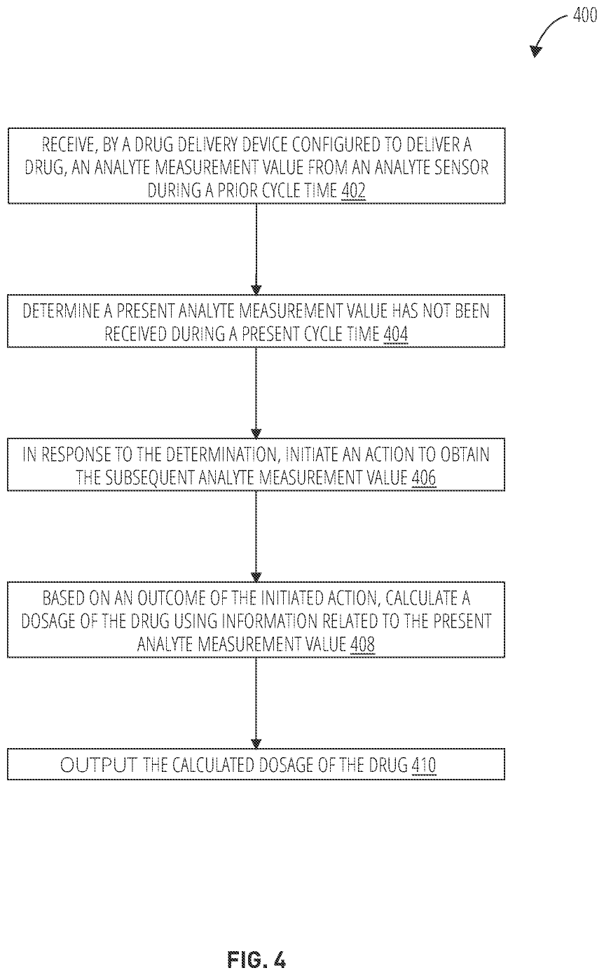

FIG. 4 illustrates a process in accordance with an embodiment that is implementable by any of the aspects illustrated in FIGS. 1-3 and 6. A drug delivery device, such as those described with reference to FIGS. 1-3, may include a processor operable to execute an automatic analyte control application. In block 402, the processor of the drug delivery device executing process 400 may receive an analyte measurement value from an analyte sensor during a prior cycle time. An example of an analyte measurement value is a blood glucose measurement value, and examples of drugs deliverable by the drug delivery device may include insulin, glucagon, or a glucagon-like peptide.

As discussed with reference to FIGS. 1-3, an analyte sensor provides analyte measurement values over the course of a time period at specific times during the time period. In the examples, when the analyte sensor is operating properly (e.g., full power supply, completed wireless connectivity, detector operation is proper, and whatever other functions or supplies that enable the analyte sensor to detect and output an analyte measurement value), the analyte measurement values are output from the analyte sensor to whichever devices are communicatively coupled to the analyte sensor.

In block 404, the processor executing process 400 may determine a present analyte measurement value has not been received during a present cycle time. If the present analyte measurement value has not been received during a present cycle time, the present analyte measurement value is considered missed or missing. For example, a missed analyte measurement value may refer to an analyte measurement value that an analyte control algorithm determines has not been received by it. A “missed analyte measurement value” may refer to an analyte measurement value that an analyte control algorithm determines has not been received by it. The analyte sensor is configured to output an analyte measurement value at a predetermined cadence or once within a set period of time for receipt by the analyte control algorithm as well as other applications, such as an artificial pancreas application, blood glucose monitoring application, or the like. In the example, the analyte sensor is configured to output an analyte measurement value at a predetermined cadence or once within a set period of time for receipt by the analyte control algorithm as well as other applications, such as an artificial pancreas application, blood glucose monitoring application, or the like. The processor may determine the present analyte measurement value has not been received when a deadline for receipt of the analyte measurement value has passed (e.g., 100 or 200 milliseconds, or the like).

In block 406, the processor executing process 400 in response to the determination, may be operable to initiate an action to obtain the present analyte measurement value. Different actions may be initiated by the processor to obtain the present analyte measurement value.

In an example of actions that may be initiated, the processor may be operable to confirm a communication connection with the analyte sensor is established and meets known requirements according to a communication protocol, such as Bluetooth or the like. For example, the communication connection may be established via a communication link, such as 214, 208 or 210. Upon confirmation of a communication connection with the analyte sensor, the processor may request the analyte sensor to send (or resend) the present analyte measurement value. In response to the request, the processor may receive the present analyte measurement value, and proceed to block 408 to continue with process 400.

In another example of the actions to be initiated, the process may be operable to confirm an absence of a communication connection with the analyte sensor. Upon confirmation of the absence of the communication connection with the analyte sensor, the processor may send signals to the analyte sensor to establish a new communication connection. The processor, upon establishment of the new communication connection, may be operable to request the analyte sensor to send the present analyte measurement value. The processor may receive the present analyte measurement value in response to the request.

As in earlier examples, after confirming the communication connection with the analyte sensor, a request may be sent to the analyte sensor. In a further example of a response to a request that the analyte sensor send the present analyte measurement value, the processor of the drug delivery device may receive, from the analyte sensor, an indication that the present analyte measurement value is not available in response to the request.

In yet a further example, the processor of the drug delivery device executing method may also when initiating actions to obtain the present analyte measure value be operable to establish contact with a controller or another device, such as a smart accessory device, cloud-based services or the like, that is communicatively coupled to the analyte sensor. The established contact may include a request sent or enable a request to be sent over a communication link, such as communication link 214 of FIG. 2 or another communication link or combination of communication links, that requests the present analyte measurement value from the controller. The processor of the drug delivery device may receive the present analyte measurement value in response to the request.

In block 408, the processor executing process 400 based on an outcome of the initiated action, calculating a dosage of the drug using information related to the present analyte measurement value.

The processor when calculating the dosage of the drug using information related to the present analyte measurement value may be operable to extract the present analyte measurement value and information related to the present analyte measurement value. The present analyte measurement value may be used in an algorithm to determine the dosage of the drug. For example, the algorithm may be implemented in the Automatic Analyte Control (AAC) 324 or an artificial pancreas application executing on a controller or on a drug delivery device.

Alternatively, the processor when calculating a dosage of the drug using information related to the present analyte measurement value, may obtain an estimate of the present analyte measurement value. The estimated present analyte measurement value may be information related to the present analyte measurement value. The estimated present analyte measurement value may be used in an algorithm to determine the dosage of the drug. The algorithm may be implemented in the Automatic Analyte Control (AAC) 324 or an artificial pancreas application executing on a controller or on a drug delivery device.

In block 410, the processor executing process 400 may be operable to cause output of the calculated dosage of the drug from the drug delivery device. For example, the processor of the drug delivery device and may be operable to send command signals to a pump mechanism to cause the output of the calculated dosage of the drug.

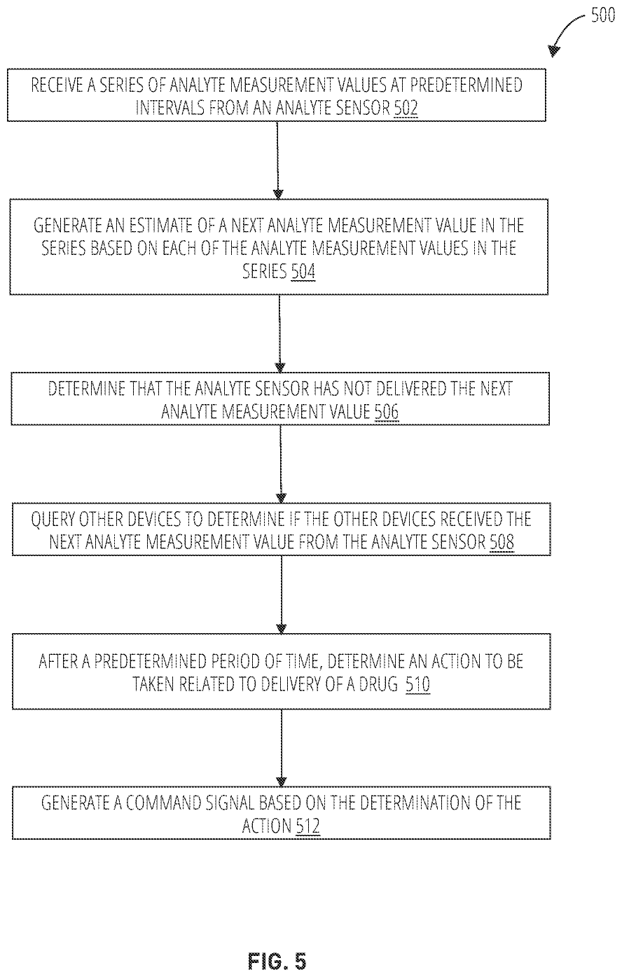

FIG. 5 illustrates another process in accordance with another embodiment that is implementable by any of the aspects illustrated in FIGS. 1-3 and 6.

In block 502, process 500 receives a series of analyte measurement values at predetermined intervals from an analyte sensor.

In block 504, process 500 generates an estimate of a next analyte measurement value in the series based on the analyte measurement values in the series. For example, the method may also include generating an estimate of a next analyte measurement value in the series based on the analyte measurement values in the series, includes obtaining each analyte measurement value and a timestamp associated with each respective analyte measurement value, estimating, based on the timestamps associated with each of the respective analyte measurement values and each of the respective analyte measurement values, a next analyte measurement value that corresponds to a time later than the latest timestamp associated with the latest respective analyte measurement value, and storing the estimated analyte measurement value as the next analyte measurement value in a memory. Each analyte measurement value may be received by a communication device coupled to the processor. In addition, each analyte measurement message may include information related to a present analyte measurement value (such as a trend indicator) as well as information related to analyte measurement values sent in previous analyte measurement values. Alternatively, the process may omit calculating the estimate until after a determination is made that the analyte sensor has not delivered the nest analyte measurement value.

In block 506, process 500 determines that the analyte sensor has not delivered the next analyte measurement value. The determination that the analyte sensor has not delivered the next analyte measurement value may include monitoring a communication device for receipt of the next analyte measurement message from an analyte sensor, determining a preset period of time has passed since a deadline for receiving the next analyte measurement value was missed, and generating a flag indicating the next analyte measurement value is missing. In an example, the preset period of time may be several milliseconds, several seconds or one to three cycles, where a cycle may include time for receipt of an analyte measurement value, determination of a dosage of a drug and delivery of the determined dosage of the drug. Additionally, or alternatively, “cycle” may refer to a sequence of operations that occur at a specified cadence that may include an establishment of a communication session between an analyte sensor and a drug delivery device of the drug delivery system, a measurement of an analyte, exchange of information of between the analyte sensor and the drug delivery device, a delivery of a dosage of a drug by the drug delivery device, and termination of the communication session.

In block 508, process 500 queries other devices to determine if the other devices received the next analyte measurement value from the analyte sensor. For example, a processor executing on the drug delivery device may, in response to a flag indicating the next analyte measurement value is missing, establish a wireless communication link with a controller. The processor may send a request for the next analyte measurement value via an established wireless communication link to the controller, cloud based services, a smart accessory device or the like. The processor may start a clock to measure an amount of time since sending the request.

In addition, or alternatively, the processor of a drug delivery device may have a data priority table indicating a prioritized order in which devices that may be communicatively coupled to the drug delivery device are to be contacted with a request. In an example, the data priority table may indicate that the drug delivery device is to connect sequentially (each one multiple times, such as 2, 3, or 5 times) with a controller executing on a smartphone, a processor of a fitness device, a processor smartwatch, and so on. In another alternative, the data priority table may indicate that the drug delivery device is to connect to any device that it is permitted to connect substantially all at the same time. Additionally, or alternatively, the processor of the drug delivery device may maintain a connection to a last good connection point. The drug delivery device and the analyte sensor communicate most effectively when the devices are in line of sight of one another. For example, placement on the body of a user is most optimal when on the same side of the body, such as inner upper, left arm and left side abdomen. In addition, when a controller when implemented on a smartphone may be able to communicate with the analyte sensor during every cycle because the smartphone likely has better quality communication components than the drug delivery device and analyte sensor, and even, perhaps, the fitness device.

In block 510, process 500 after a predetermined period of time, the processor may determine an action to be taken related to delivery of a drug. For example, the processor may as part of the action receive a response to the query from one of the other devices, such as a controller, a smart accessory device, a cloud-based service, or the like. The response may include the next analyte measurement value that was missing as well as other information related to the next analyte measurement value. Alternatively, the response may indicate how the processor may obtain or retrieve the next analyte measurement value. The next analyte measurement value and the other information related to the next analyte measurement value may be used in block 512 in the generation of a command signal for outputting a next dosage of the drug. For example, the next analyte measurement value may be used to calculate the next dosage of the drug.

Alternatively, the method, when determining an action to be taken related to delivery of a drug may use the generated estimate of the next analyte measurement value, may in response to receiving an indication that the next analyte measurement value is not available, retrieve the estimate of the next analyte measurement value from the memory. The processor may determine a next dosage of a drug to be delivered based on the retrieved estimate of the next analyte measurement value and indicate output of the determined next dosage of the drug as the determination of the action.

Alternatively, at block 510, the processor may, in response to the next analyte measurement value being received in response to the request, determine a next dosage of a drug to be delivered from a reservoir of the drug delivery device using the next analyte measurement value, and indicating output of the determined next dosage of the drug as the determination of the action.

In block 512, process 500 generates a command signal based on the determination of the action. For example, when the processor determines the next dosage, the determined action may be to generate a command signal for outputting a dosage of the drug. In response, the processor may generate a command signal to be delivered to a pump mechanism coupled to the reservoir of the drug delivery device and communicatively coupled to the processor. The command signal, for example, may include instructions related to the determined next dosage of the drug. The processor may be further operable to output the command signal to the pump mechanism for delivery of the next dosage of the drug.

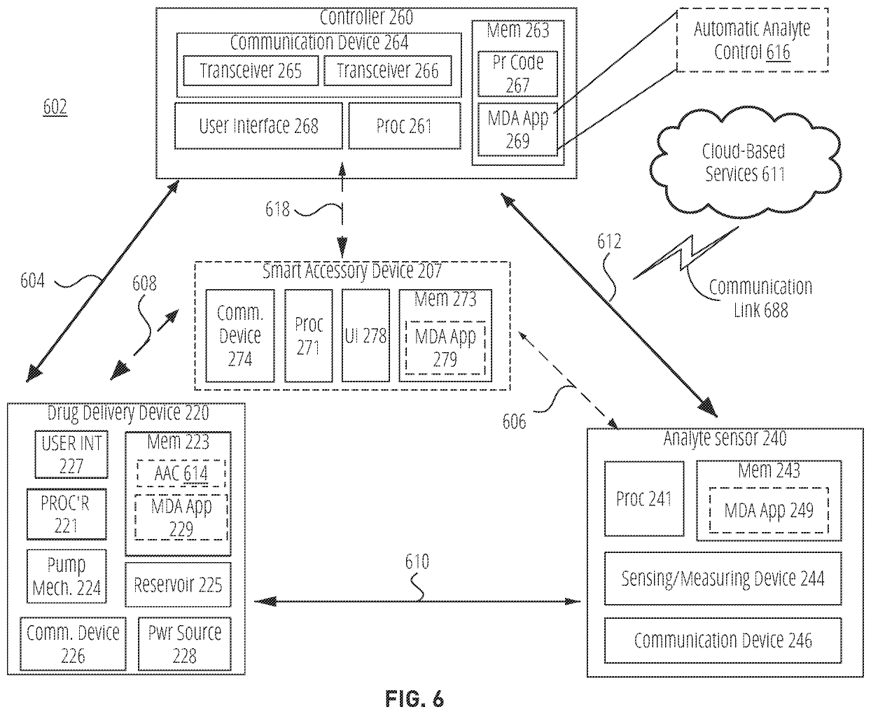

FIG. 6 illustrates a functional block diagram of a system example suitable for implementing the example processes and techniques described herein.

The drug delivery system environment 602 may be include components such as an automatic drug delivery system that is configured to determine a drug dosage and deliver or output, the dosage of the drug without any user interaction, or in some examples, limited user interaction, such as in response to a user depressing a button to indicate measurement of blood glucose or another analyte, or the like. A “drug delivery system environment” may refer to a computing and sensing environment that includes cloud based services, a drug delivery system (that may include a controller, a drug delivery device, and an analyte sensor) and optionally additional devices. The components of the drug delivery system environment may cooperate to provide present analyte measurement values or at least accurate estimates of present analyte measurement values to facilitate calculation of optimal drug dosages for a user.

The drug delivery system environment 602, in some examples, may include a controller 260, a drug delivery device 220, an analyte sensor 240, and cloud-based services 611. In another example, the drug delivery system environment 602 may include a controller 260, a drug delivery device 220, an analyte sensor 240, cloud-based services 611 as well as the smart accessory device 207.

Different systems or devices of the drug delivery system environment 602 may implement (and/or provide functionality for) a medication delivery algorithm or application (MDA). An example of an MDA may be an artificial pancreas (AP) application that may be configured to govern or control automated delivery of a drug or medication, such as insulin, to a user (e.g., to maintain euglycemia—a normal level of glucose in the blood). The MDA may, for example, receive information from additional applications or algorithms that execute on a device within the drug delivery system environment 602.

The drug delivery device 220 may be configured to perform and execute the processes described in the examples of FIGS. 1-5 without input from the controller 260 or the optional smart accessory device 207. The drug delivery device 220 may include a user interface 227, a processor (PROC'R) 221, a pump mechanism 224, a communication device 226, a memory 223, a power source 228, and a reservoir 225.

The processor 221 alone may implement the processes to determine a response to a determination of a missing blood glucose measurement value as described with respect to the other examples. The processor 221 of the drug delivery device 220 may be operable to implement delivery of a drug to the user according to a diabetes treatment plan or other drug delivery regimen stored in the memory 223. For example, the processor 221 may be operable to execute programming code and be configured when executing non-transitory programming code of a medication delivery application or algorithm, such as MDA APP 229 and other programs, such as an algorithm 262, to perform the functions that implement the example routines and processes described herein. In an operational example, the processor 221, when executing the programming code implementing MDA APP 229, may be configured to output a control signal causing actuation of the pump mechanism 224 to deliver drug dosages or the like as described with reference to the examples of FIGS. 1-5.

The memory 223 may store programming code executable by the processor 221. The programming code, for example, may enable the processor 221 to control expelling insulin from the reservoir 225 and control the outputting of doses of medication based on signals from the MDA APP 229 or, external devices, when the drug delivery device 220 is configured to receive and respond to the external control signals and be operable to deliver a drug based on information received from the analyte sensor 240, the cloud-based services 211 and/or the controller 260 or optional smart accessory device 207. The memory 223 may also be configured to store other data and programming code, such as the automatic analyte control application 614.

The reservoir 225 may be configured to store drugs, medications or therapeutic agents suitable for automated delivery, such as insulin, glucagon, glucagon-like peptide, morphine, blood pressure medicines, chemotherapy drugs, a combination of these drugs or agents, or the like.

In an example, the drug delivery device 220 includes a communication device 226, which may be a receiver, a transmitter, or a transceiver that operates according to one or more radio-frequency protocols, such as Bluetooth, Wi-Fi, a near-field communication standard, a cellular standard, or the like. The processor 221 in addition communicating with the smart accessory device 207 may, for example, communicate with the controller 260 and an analyte sensor 240 via the communication device 226.

When configured to communicate with an external device, such as the controller 260 or the analyte sensor 240, the drug delivery device 220 may receive signals over the communication link 604 from the controller 260 or wireless communication link 612 from the analyte sensor 240. The processor 221 of the drug delivery device 220 may receive and process the signals from the respective external devices (e.g., cloud-based services 611, smart accessory device 207, or controller 260) to implement (or modify) delivery of a drug to the wearer in response to signals received from the analyte sensor 240.

The processors described herein may be integrated circuits (ICs), application specific ICs (ASICs), field programmable arrays (FPGAs), and/or programmable logic devices (PLDs), or the like.

The processor 221 when executing the MDA APP 229 may output a control signal operable to actuate the pump mechanism 224 to deliver a drug, such as insulin, glucagon, glucagon-like peptide, or a combination thereof, in response to a determination of a user exercising.

The drug delivery device 220 may be a wearable automatic drug delivery device that may be attached to the body of a user (i.e., a wearer), such as a patient or diabetic, at an attachment location via an adhesive layer (not shown) and may deliver any therapeutic agent, any drug or medicine, such as insulin or the like, to a user at or around the attachment location.

The drug delivery device 220 may, for example, include a reservoir 225 configured to store the drug, a needle or cannula (not shown) may be fluidically coupled to the reservoir and may be subcutaneously, intraperitoneally, or intravenously coupled via a needle/cannula (not shown) to a user's body. Some components of the pump mechanism 224 may be coupled to reservoir 225 and the needle/cannula. Additionally, the pump mechanism 224 may be communicatively coupled to the processor 221. The pump mechanism 224 may be configured to enable transfer the drug from the reservoir 225 through the needle or cannula and into the user.

The drug delivery device 220 may further include a power source 228, such as a battery, a piezoelectric device, other forms of energy harvesting devices, or the like, for supplying electrical power to the pump mechanism 224 and/or other components (such as the processor 221, memory 223, and the communication device 226). Different techniques may be used to obtain harvest energy such as piezoelectric transducers, and the like for storage as electrical energy in the power source 228.

The smart accessory device 207, may be a device such as a smartwatch, a personal assistant device or the like, which may communicate with the other components of drug delivery system environment 602 via either a wired or wireless communication links 606, 608 or 618. The smart accessory device 207 may be, for example, an Apple Watch®, other wearable smart device, including eyeglasses, provided by other manufacturers, a global positioning system-enabled wearable, a wearable fitness device, smart clothing, or the like. Like the controller 260, the smart accessory device 207 may also be configured to perform various functions including controlling the drug delivery system 816. For example, the smart accessory device 207 may include a communication device 274, a processor 271, a user interface 278, and a memory 273. The user interface 278 may be a graphical user interface presented on a touchscreen display of the smart accessory device 207. The processor 271 that may execute programming code, such as the MDA APP 279 for controlling the wearable automatic drug delivery device 220 to implement the examples described with reference to FIG. 1-5.

The controller 260 may be a computing device such as a smart phone, a tablet, a personal diabetes controller, a dedicated diabetes therapy controller, or the like. In an example, the controller 260 may include a processor 261, a controller memory 263, a user interface 268, and a communication device 264. The controller 260 may contain analog and/or digital circuitry that may be implemented as a processor 261 for executing processes based on programming code stored in the controller memory 263, such as the MDA algorithm or application (APP) 269 or the Automatic Analyte Control 616, that may be configured to manage a response to a missed analyte measurement value from the analyte sensor 240. The controller 260 may be used to initially set up, adjust settings, and/or control operation of the wearable automatic drug delivery device 220 and/or the analyte sensor 240 as well as the optional smart accessory device 207.

The processor 261 may also be configured to execute programming code stored in the controller memory 263, such as programming code 267 and the MDA application 269. The MDA application 269 may include programming code that is operable upon execution by the processor 261 to provide estimated analyte measurement values or missed analyte measurement values as well process signals received from the analyte sensor 240 and may be operable manage liquid drug delivery based on determinations from the Automatic Analyte Control 616 and the MDA application 269.

The user interface 268 may be under the control of the processor 261 and be configured to present a graphical user interface that enables the presentation of analyte measurement values, notifications or alarms related to the of a meal announcement, adjust setting selections and the like as described above.

The communication device 264 may include one or more transceivers, such as transceiver 265 and transceiver 266, and receivers or transmitters that operate according to one or more radio-frequency protocols. In the example, the transceivers 265 and 266 may be a cellular transceiver and a Bluetooth® transceiver, respectively. For example, the transceivers 265 and 266 may be configured to receive and transmit signals containing information usable by the MDA APP 269.

In some examples, the controller 260 may include a user interface 268, respectively, such as a keypad, a touchscreen display, levers, light-emitting diodes, buttons on a housing (shown in another example) of the controller 260, a microphone, a camera, a speaker, a display, or the like, that is configured to allow a user to enter information and allow the controller 260 to output information for presentation to the user (e.g., alarm signals or the like). The user interface 268 may provide inputs, such as a voice input, a gesture (e.g., hand or facial) input to a camera, swipes to a touchscreen, or the like, to processor 261 which the programming code interprets.

The analyte sensor 240 may include a processor 241, a memory 243, a sensing/measuring device 244 and a communication device 246. The analyte sensor 240 may be communicatively coupled to the processor 261 of the controller or processor 221 of the wearable automatic drug delivery device 220. The memory 243 of the analyte sensor 240 may be configured to store information and programming code, such as an instance of the MDA APP 249.

The analyte sensor 240 may be configured to detect multiple different analytes, such as lactate, ketones, uric acid, sodium, potassium, alcohol levels, hormone levels, or the like, and output results of the detections, such as measurement values or the like. The analyte sensor 240 may, in an example, be configured to measure a blood glucose value at a predetermined time interval, such as every 5 minutes, or the like. The communication device 246 of analyte sensor 240 may have circuitry that operates as a transceiver for communicating the measured blood glucose values to the controller 260 over a wireless link 714 or with wearable automatic drug delivery device 220 over the wireless communication link 604. While called an analyte sensor 240, the sensing/measuring device 244 of the analyte sensor 240 may include one or more additional sensing elements, such as a glucose measurement element, a hormone detection element, a heart rate monitor, a pressure sensor, or the like. The processor 241 may include discrete, specialized logic and/or components, an application-specific integrated circuit, a microcontroller or processor that executes software instructions, firmware, programming instructions stored in memory (such as memory 243), or any combination thereof.

Like the processor 221, the processor 241 of the analyte sensor 240 may be operable to perform many functions. For example, the processor 241 may be configured by the programming code stored in the memory 243 to manage the collection and analysis of data detected the sensing and measuring device 244 and deliver the results of the analysis and/or the data to the controller 260, the drug delivery system 816, or both.

Although the analyte sensor 240 is depicted in FIG. 6 as separate from the wearable automatic drug delivery device 220, in various examples, the analyte sensor 240 and wearable automatic drug delivery device 220 may be incorporated into the same unit. That is, in various examples, the sensor 240 may be a part of the wearable automatic drug delivery device 220 and contained within the same housing as the wearable automatic drug delivery device 220. In an example, the sensor 240 or, only the sensing/measuring device 244 and memory storing related programming code may be positioned within or integrated into, or into one or more components, such as the memory 243 of, the wearable automatic drug delivery device 220. In such an example configuration, the processor 241 may be able to implement the process examples of FIGS. 1A-2 alone without any external inputs from the controller 260, the cloud-based services 211, the optional smart accessory device 207, or the like.

The communication link 688 that couples the cloud-based services 611 to the respective devices 220, 240, 260 or 207 of drug delivery system environment 602 may be a cellular link, a Wi-Fi link, a Bluetooth link, or a combination thereof. Services provided by cloud-based services 611 may include data storage that stores anonymized data, such as blood glucose measurement values, drug delivery history, bolus delivery history, time data, and other forms of data. In addition, the cloud-based services 611 may process the anonymized data from multiple users to provide past and present analyte measurement values (including blood glucose measurement values) as well as generalized information related to clinical diabetes-related data and the like.

The wireless communication links 604, 606, 608, 610, 612, 618 and 688 may be any type of wireless link operating using known wireless communication standards or proprietary standards. As an example, the wireless communication links 604, 606, 608, 610, 612, 618 and 688 may provide communication links based on Bluetooth®, Zigbee®, Wi-Fi, a near-field communication standard, a cellular standard, or any other wireless protocol via the respective communication devices 226, 246, 264 and 274.

Software related implementations of the techniques described herein, such as the processes examples described with reference to FIGS. 1-5 may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

What is claimed is:

1. A drug delivery device, comprising:
a processor; and
a memory storing instructions that, when executed by the processor, configure the drug delivery device to:
receive an analyte measurement value from an analyte sensor during a prior cycle time;
determine a present analyte measurement value has not been received during a present cycle time, wherein the present analyte measurement value is received after the analyte measurement value received during the prior cycle time;
in response to the determination, initiate an action to obtain the present analyte measurement value; and
based on an outcome of the initiated action, calculate a dosage of the drug using the present analyte measurement value; and
output the calculated dosage of the drug.

2. The drug delivery device of claim 1, wherein the processor, when initiating the action to obtain the present analyte measure value, is further configured to:
confirm a communication connection with the analyte sensor;
upon confirmation of the communication connection with the analyte sensor, request the analyte sensor to send the present analyte measurement value; and
receive the present analyte measurement value in response to the request.

3. The drug delivery device of claim 2, wherein the processor, when calculating the dosage of the drug using the present analyte measurement value, is further configured:
extract the present analyte measurement value and information related to the present analyte measurement value; and
use the present analyte measurement value in an algorithm to determine the dosage of the drug.

4. The drug delivery device of claim 1, wherein the processor, when initiating the action to obtain the present analyte measure value, is further configured to:
confirm an absence of a communication connection with the analyte sensor;
upon confirmation of the absence of the communication connection with the analyte sensor, send signals to the analyte sensor to establish a new communication connection; and
upon establishment of the new communication connection, request the analyte sensor to send the present analyte measurement value; and
receive the present analyte measurement value in response to the request.

5. The drug delivery device of claim 1, wherein the processor when initiating the action to obtain the present analyte measure value is further configured to:
establish contact with a controller that is communicatively coupled to the analyte sensor; and
request the present analyte measurement value from the controller; and
receive the present analyte measurement value in response to the request.

6. The drug delivery device of claim 1, wherein the processor when initiating actions to obtain the present analyte measure value is further configured to:
confirm a communication connection with the analyte sensor;
upon confirmation of a communication connection with the analyte sensor, request the analyte sensor to send the present analyte measurement value; and
receive an indication that the present analyte measurement value is not available in response to the request.

7. The drug delivery device of claim 6, wherein the processor when calculating the dosage of the drug use information related to the present analyte measurement value, is further configured to:
obtain an estimate of the present analyte measurement value, wherein the estimated present analyte measurement value is the information related to the present analyte measurement value; and
use the estimated present analyte measurement value in an algorithm to determine the dosage of the drug.

8. The drug delivery device of claim 1, wherein the processor when calculating the dosage of the drug using information related to the present analyte measurement value, is further configured to:
obtain an estimate of the present analyte measurement value, wherein the estimated present analyte measurement value is the information related to the present analyte measurement value; and
use the estimated present analyte measurement value in an algorithm to determine the dosage of the drug.

9. The drug delivery device of claim 1, further comprising:
a reservoir configured to store the drug; and
a pump mechanism operable to output the drug in response to command signals from the processor.

10. A method, comprising:
receiving, by a processor of a drug delivery device, a series of analyte measurement values at predetermined intervals from an analyte sensor;
generating an estimate of a next analyte measurement value in continuation of the series based on each of the analyte measurement values in the series;
determining that the analyte sensor has not delivered the next analyte measurement value;
querying other devices to determine if the other devices received the next analyte measurement value from the analyte sensor;
after a predetermined period of time, determining an action to be taken related to delivery of a drug using the estimate of the next analyte measurement value; and
generating a command signal based on the determination of the action.

11. The method of claim 10, wherein the generating the estimate of the next analyte measurement value in the series based on the analyte measurement values in the series, comprises:
obtaining a timestamp associated with each analyte measurement value of the analyte measurement values;
estimating, based on the timestamp associated with each analyte measurement value and the analyte measurement value, a next analyte measurement value that corresponds to a time later than a latest timestamp associated with a latest respective analyte measurement value; and
storing the estimated analyte measurement value in a memory.

12. The method of claim 11, wherein determining the action to be taken related to delivery of the drug using the generated estimate of the next analyte measurement value, comprises:
in response to receiving an indication that the next analyte measurement value is not available, retrieving the estimate of the next analyte measurement value from the memory;
determining a next dosage of a drug to be delivered based on the retrieved estimate of the next analyte measurement value; and
indicating output of the determined next dosage of the drug as the determination of the action.

13. The method of claim 10, wherein determining that the analyte sensor has not delivered the next analyte measurement value, comprises:
monitoring a communication device for receipt of the next analyte measurement value from the analyte sensor;
determining a preset period of time has passed since a deadline for receiving the next analyte measurement value was missed; and
generating a flag indicating the next analyte measurement value is missing.

14. The method of claim 13, wherein the preset period of time is several milliseconds, several seconds or one to three cycles, and a cycle includes time for receipt of an analyte measurement value, determination of a dosage of a drug and delivery of a dosage of the drug.

15. The method of claim 10, wherein the next analyte measurement value includes information related to the next analyte measurement value as well as information related to previously-sent analyte measurement values.

16. The method of claim 10, wherein the querying other devices to determine if the other devices received the next analyte measurement value from the analyte sensor, comprises:
in response to a flag indicating the next analyte measurement value is missing, establishing a wireless communication link with a controller;
sending a request via the established wireless communication link to the controller, wherein the request is for the next analyte measurement value; and
starting a clock to measure an amount of time since sending the request.

17. A drug delivery system, comprising:
a controller including a processor, a communication device, and a memory, wherein the memory stores programming code and an automatic analyte control application;
a drug delivery device including a drug delivery device processor, a drug delivery device memory, a drug delivery device communication device, a reservoir, and a pump mechanism; and
an analyte sensor including an analyte sensor processor, an analyte sensor communication device, and a sensing/measuring device, the analyte sensor is configured to periodically obtain, via the sensing/measuring device, an analyte measurement value of an analyte from a user of the drug delivery system,
wherein the drug delivery device processor is operable to:
receive an analyte measurement value from the analyte sensor during a prior cycle time;
determine a present analyte measurement value has not been received during a present cycle time;
in response to the determination, initiate an action to obtain the present analyte measure value; and
based on an outcome of the initiated action, calculate a dosage of a drug using information related to the present analyte measurement value; and
output the calculated dosage of the drug.

18. The drug delivery system of claim 17, wherein the initiating the action to obtain the present analyte measure value further comprises:
confirm a communication connection with the analyte sensor;
upon confirmation of a communication connection with the analyte sensor, requesting the analyte sensor to send the present analyte measurement value; and
receive the present analyte measurement value in response to the request.

19. The drug delivery system of claim 18, wherein the calculating of the dosage of the drug using information related to the present analyte measurement value, comprises:
extract the present analyte measurement value and information related to the present analyte measurement value; and
use the present analyte measurement value in an algorithm to determine the dosage of the drug.

20. The drug delivery system of claim 17, wherein the initiating the action to obtain the present analyte measure value further comprises:
confirm an absence of a communication connection with the analyte sensor;
upon confirmation of the absence of the communication connection with the analyte sensor, sending signals to the analyte sensor to establish a new communication connection; and
upon establishment of the new communication connection, request the analyte sensor to send the present analyte measurement value; and
receive the present analyte measurement value in response to the request.
